(19) United States
(12) Patent Application Publication
(10) Pub. No.: US 2026/0085265 A1
(43) Pub. Date: Mar. 26, 2026

(54) POLYMERIC MULTILAYER FILM FOR USE IN BIOPROCESSING APPLICATIONS

(71) Applicant: Sealed Air Corporation (US), Charlotte, NC (US)

(72) Inventors: Mahdy Moghani, Charlotte, NC (US); William Peyton Roberts, Charlotte, NC (US); Zach Cogan, Charlotte, NC (US); Bernice Karlton-Senaye, Concord, NC (US)

(21) Appl. No.: 19/109,396

(22) PCT Filed: Oct. 9, 2023
(86) PCT No.: PCT/US2023/034737

§ 371 Date: Mar. 6, 2025

(30) Foreign Application Priority Data
Oct. 11, 2022 (EP) 22200704.9

(51) Int. Cl. C12M 1/00; B32B 27/08; B32B 27/20; B32B 27/30; B32B 27/32; B32B 27/34; B32B 27/36

(52) U.S. Cl. CPC C12M 23/20; B32B 27/08; B32B 27/20; B32B 27/306; B32B 27/32; B32B 27/34; B32B 27/36; C12M 23/26; B32B 2250/24; B32B 2264/105; B32B 2307/72; B32B 2307/7244; B32B 2307/7376; B32B 2439/00

(57) ABSTRACT

The presently disclosed subject matter is directed to a polymeric, multilayer film suitable for use in a variety of applications, including (but not limited to) the manufacture of bioprocessing containers. The disclosed film comprises a sealant layer comprising: (i) one or more ethylene/alpha olefin copolymers with an average density lower than 0.922 g/cc; (ii) one or more polymers with an average density higher than or equal to 0.950 g/cc and selected among ethylene/alpha olefin copolymers and/or ethylene homopolymers; (iii) high molecular weight siloxane polymers; optionally, at least one gas barrier layer and an outer skin layer. The sealant layer of the disclosed films provides for a low blocking effect and for high seal strength. In addition, the surface of the sealant layer is inert and substantially free from migratory surface additives and from metal containing particulate additives, such that, when the film is used for manufacturing bioprocessing containers, cell growth and products harvesting are not affected. A method for manufacturing the film, a bioprocessing container comprising the film and a method for culturing cells in such container are also herein disclosed.

### FIELD OF THE INVENTION

The presently disclosed subject matter generally relates to polymeric films suitable for use in manufacturing disposable flexible containers for bioprocessing applications (“bioreactors” or “bioprocessing containers”). More particularly, the presently disclosed films allow to manufacture bioreactors which have a biologically inert internal surface, which does not affect cell growth and the collection of the bioprocessing products (cell cultures, cellular aggregates, particles, tissues, proteins, drugs, and the like) inside them and which are endowed with high seal performance when in use.

### BACKGROUND

The development and commercialization of many processes in the fields of medicine, chemistry, and agriculture require the use of bioprocessing containers (bioreactors). Cells have typically been grown in vitro in glass, metal, or hard plastic vessels. However, because these culture vessels are not disposable, they are expensive and require maintenance. Particularly, to maintain a sterile or aseptic environment for cell culture, the vessels require sterilization, usually by autoclave or aseptic disinfection. Thus, they must be washed and sterilized prior to and/or subsequent to use. The expense of producing cells, biopharmaceuticals, biologicals, and the like is often exacerbated by the required cleaning, sterilization, and validation of conventional bioprocessing containers (i.e., metal, glass, or hard plastic vessels). In addition, because glass, metal, and hard plastic vessels are not disposable, it is necessary to have a large amount of space to accommodate storage.

Attempts have been made to solve these problems with the development of pre-sterilized disposable bioprocessing containers manufactured from flexible, multilayer polymeric films.

Films to be used in manufacturing disposable bioreactors are typically manufactured in a tubular form, for example they are blown or round cast. Manufacturing the films in tubular form offers the advantage that the sealant layer (which will form the internal surface of the disposable bioreactor) is on the inside of the tube and does not contact any solid or liquid surfaces after the films exits the die opening, only coming into contact with air. The air in the inside of the tube can be controlled to have very low particle count through technologies known in the art. Accordingly, foreign contaminations of the sealant layer of the films are minimized. The film in tubular form is then flattened to form a two-ply flattened tube where the inside sealant layers of the two plies contact each other. This two-ply form in which the sealant layers of the two plies are in contact with each other will then protect such layers of the films from any contamination.

However, the two-ply form presents the drawback that the sealant layers of the two plies may adhere to one another and separation of the plies at the customer facility (e.g. in order to manufacture the disposable bioreactors) may be difficult. To avoid this issue (commonly referred to as “blocking”), for applications where inertness of the surface is not mandatory (such as food packaging), slip agents (typically amide waxes such as euracamide) and antiblock agents (typically in the form of inorganic solid particles), are incorporated in the sealant layer of films manufactured in tubular form, during extrusion. However, slip agents like waxes and inorganic solid particles are undesired in films for bioprocessing applications. Amide waxes are migratory slip additives, which due to their low molecular weight and polarity, can migrate through the film, bloom to the surface thereof and even detach from the film surface and drop into the content of the bioreactor or promote adhesion of the cultured cells to the surface of the film. This, in turn, may impact yield when suspended cell culture is performed in the bioreactor (i.e. when the cell culture is non-adherent). Inorganic solid particles are undesired because generally comprise metallic elements such as magnesium, calcium, aluminum, whose presence is tightly controlled in bioprocessing applications as they can disrupt cell growth or interfere with biological processes.

For this reason, in films for bioprocessing applications it is preferable to avoid the presence of migratory additives such as amide waxes and of inorganic solid particles and to have a high molecular weight, non-polar polymeric composition for the sealant layer which allows a smooth separation of the plies when the two-ply flat tube has to be enlarged.

U.S. Pat. No. 8,722,164 in the name of Bekele at al. discloses the use of olefin hydrocarbon polymer with glass transition temperature (Tg) higher than or equal to 25° C. along with alpha-olefin copolymer in the sealant layer of films to be used for manufacturing disposable bioreactors. Cyclic olefin copolymers (COC) with Tg≥25° C. are typically used as the olefin hydrocarbon polymers. Disposable bioreactors are manufactured by sealing two or more films through their sealant layers to form a container. The presence of COC in the sealant layer has shown to reduce the seal strength of the sealed films when they have been subjected to moderately high temperatures, for example temperatures higher than 60° C., which may occur during shipping or storage of the films. The reduction in seal strength can be avoided by extending the sealing time, which however has the drawback of increasing time and costs involved in the sealing process.

The reduced seal strength may cause leakages or even the opening of the container, which results in contaminations and/or loss of the biological material from the bioprocess. Specifically, this drawback limits the use of such films for large size bioreactors where the seals are subjected to high pressure for an extended period of time during the bioprocess.

One alternative to the above problems may reside in manufacturing the films for bioprocessing applications in a single-ply form rather than a tubular, two-ply form. The single-ply form does not require a specific formulation of the sealant layer to allow plies separation. On the other hand, processes to manufacture the films in single-ply form (such as flat cast) will inevitably expose the sealant layer to direct or indirect contact with the manufacturing equipment (e.g. rollers), with consequent, unavoidable foreign contaminations.

Therefore, there remains the need for a polymeric film suitable for bioprocessing applications which has some key properties, namely:
its sealant layer (which finally forms the internal surface of the bioreactor, which contacts the hosted material) has low interaction with the material inside the bioreactor (liquid medium, cultured cells, products from the bioprocessing, housed biological fluids, etc.). This feature (generally known as inertness) allows for a high yield of cell growth, ease of harvesting of non-adherent cell cultures (where cells are suspended in the liquid medium) or of products from the bioprocessing and avoids contamination of the material inside the bioreactor;
its sealant layer provides high seal strength which remains stable even if the film undergoes exposure to high temperatures, thus allowing the manufacture of bioreactors with a high load-carrying capacity and/or capable of facing internal pressure when in use;
the composition of the sealant layer allows a smooth separation of the two plies of the flattened tube, thus advantageously permitting the manufacture of the film in tubular form.

It is also advantageous that the film, and its sealant layer in particular, be substantially free from the migratory surface additives typically used in the external layers of polymeric films (such as antifogging agents, antistatic agents, slip agents such as amide waxes, protein coatings, therapeutic agent coating, binding agents and the like) and from metal containing particulate additives working as anti-blocking agents, such that the growth of cell cultures and the collection of bioprocessing material is not affected.

### SUMMARY

The presently disclosed subject matter is directed to a polymeric, multilayer film comprising:
a sealant layer comprising:
(i) 58-95.5 wt % in respect of the total weight of the sealant layer of one or more ethylene/alpha olefin copolymers having an average density lower than 0.922 g/cc;
(ii) 4-10 wt % in respect of the total weight of the sealant layer of one or more polymers having an average density higher than or equal to 0.950 g/cc and selected among one or more ethylene/alpha olefin copolymers and/or one or more ethylene homopolymers;
(iii) 0.5-2.0 wt % in respect of the total weight of the sealant layer of high molecular weight siloxane polymers;
(iv) optionally, 5-30 wt % in respect of the total weight of the sealant layer of elastomeric cyclic olefin copolymers (COC) with glass transition temperature (Tg) lower than 20° C.;
optionally, at least one gas barrier layer comprising one or more polymers selected from the group consisting of poly(glycolic acid) (PGA), polyamides, ethylene vinyl alcohol (EVOH) and mixtures thereof;
an outer skin layer comprising one or more polymers selected from the group consisting of: (co)polyesters, polyamides, high-density polyethylene (HDPE), polypropylene (PP) and their admixtures.

The presently disclosed subject matter is also directed to a bioprocessing container comprising at least a film as presently disclosed, wherein the film is sealed to define an interior compartment of the container and wherein the sealant layer of the disclosed film forms the internal surface of the container, i.e. the surface coming into contact with the material housed in the container. The presently disclosed subject matter is also directed to a method for culturing cells. The disclosed method comprises either the steps of:
(i) providing the disclosed bioprocessing container;
(ii) introducing a liquid medium into the interior compartment of the container;
(iii) inoculating the liquid medium with cells, and
(iv) incubating the cells within the interior compartment of the container under suitable conditions for cell growth;
or the steps of:
(i) providing the disclosed bioprocessing container;
(ii) pre-inoculating a liquid medium with cells;
(iii) introducing the pre-inoculated liquid medium into the interior compartment of the container, and
(iv) incubating the cells within the interior compartment of the container under suitable conditions for cell growth.

### DETAILED DESCRIPTION

### I. General Considerations

The presently disclosed subject matter is directed to a polymeric multilayer film suitable for use in a wide variety of applications, such as (but not limited to) the manufacture of bioprocessing containers. The presently disclosed film, and, in particular, the sealant layer thereof, is substantially free from migratory surface additives and from metal containing particulate additives: this results in a biologically inert internal surface of the bioreactor manufactured with such film. In addition, the composition of the sealant layer prevents adhesion between the two plies of the film (blocking) when it is manufactured in tubular form and flattened, without affecting the strength of the seals.

### II. Definitions

While the following terms are believed to be well understood by the person skilled in the art, the following definitions are set forth to facilitate explanation of the presently disclosed subject matter.

Unless defined otherwise, all technical and scientific terms used herein have the same meaning as commonly understood by the person skilled in the art to which the presently disclosed subject matter belongs.

As used herein, the term “film” is used in a generic sense to refer to a plastic web, regardless of whether it is a film or a sheet, i.e. regardless of its thickness. The term “multilayer film” as used herein refers to a thermoplastic material, generally in sheet or film form, comprising one or more layers comprising polymers and/or other materials, wherein said layers are bonded together by any conventional or suitable method, including one or more of the following: (co)extrusion, extrusion coating, lamination, vapor deposition coating, solvent coating, emulsion coating, and/or suspension coating.

As used herein, the phrase “inner layer” in connection with the multilayer film refers to a layer having both its surfaces directly adjacent to other layers of the film.

As used herein, the phrase “outer layer” in connection with the multilayer film refers to a layer having only one of its surfaces directly adjacent to another layer of the film. An outer layer defines an outer film surface.

As used herein, the term “adjacent” as applied to film layers refers to the positioning of two layers in contact with one another with or without an intervening layer (such as a tie layer), adhesive, or other layer therebetween. The term “directly adjacent” as applied to film layers refers to adjacent layers that are in contact with one another without any tie layer, adhesive, or other layer therebetween.

As used herein, the terms “major amount” or “major proportion” refer to an amount of a component higher than 50% by weight in respect of the total amount by weight of the components of a referred element (e.g. a film, a layer etc.).

As used herein, the terms “minor amount” or “minor proportion” refer to an amount of a component lower than 50% by weight in respect of the total amount by weight of the components of a referred element (e.g. a film, a layer etc.). As used herein, the phrases “seal layer”, “sealing layer”, “heat seal layer”, “sealant layer”, “heat sealable layer” and the like refer to an outer film layer, or layers, involved in the sealing of the film to itself, to another film layer of the same or another film, and/or to another article that is not a film. It should also be recognized that in general, up to the outer 25.4-254 μm (1-10 mils) of a film can be involved in the sealing of the film to itself or another layer. In general, a sealant layer sealed by heat-sealing layer comprises any thermoplastic polymer. In some embodiments, the heat-sealing layer can comprise, for example, thermoplastic polyolefins, thermoplastic polyamides, thermoplastic polyesters, and thermoplastic polyvinyl chloride. In some embodiments, the heat-sealing layer can comprise thermoplastic polyolefins such as ethylene-vinyl acetate (EVA) and polyethylene.

As used herein, the terms “skin layer” or “abuse layer” refer to the outer layer of the multilayer film that is opposite to the seal layer and that, in the final container, will be in contact with the external environment. This layer can be subject to abuse during storage and handling of the film or of the container manufactured from that film.

As used herein, the term “tie layer” refers to any inner layer having the primary function of adhering two layers to one another. In some embodiments, tie layers can comprise any nonpolar polymer having a polar group grafted thereon, such that the polymer is capable of covalent bonding to polar polymers such as polyamide, PGA, and/or ethylene/vinyl alcohol copolymer. In some embodiments, tie layers can comprise at least one member selected from the group including, but not limited to, modified polyolefin, modified ethylene/vinyl acetate copolymer, and/or homogeneous ethylene/alpha-olefin copolymer.

The term “bulk layer” or “structural layer” as used herein refers to a layer generally used to increase the abuse-resistance, puncture resistance, toughness, modulus, etc., of a film, or just to provide the desired thickness. In some embodiments, the bulk layer can comprise polyolefin, including (but not limited to) ethylene/alpha-olefin copolymers, such as linear low density polyethylene (LLDPE) or very low density polyethylenes (VLDPE), ethylene/alpha-olefin copolymer plastomers, low density polyethylene (LDPE), ethylene-vinyl acetate copolymers (EVA), ethylene/acrylic acid copolymers (EAA) or ethylene/methacrylic acid copolymers (EMAA), ethylene/methyl acrylate copolymers (EMA), ethylene/butyl acrylate copolymers (EBA), ionomers, and blends thereof.

As used herein, the terms “barrier” or “gas barrier” when referred to a layer, to a resin contained in said layer, or to a film, refer to the property of a layer, resin, or film to serve as a barrier which limits to a certain extent the passage of gases, preferably of oxygen, and/or odors through itself. Examples of polymeric materials with low oxygen transmission rates (OTR) useful in such a layer can include: ethylene/vinyl alcohol copolymer (EVOH), polyvinylidene dichloride (PVDC), vinylidene chloride copolymer such as vinylidene chloride/methyl acrylate copolymer, vinylidene chloride/vinyl chloride copolymer, polyamide, co-polyamide, poly(glycolic acid) (PGA), polyester, polyacrylonitrile (available as Barex™ resin), or blends thereof. Oxygen barrier materials can further comprise high aspect ratio fillers that create a tortuous path for permeation (e.g., nanocomposites). Oxygen barrier properties can be further enhanced by the incorporation of an oxygen scavenger, such as an organic oxygen scavenger. In some embodiments, metal foil, metallized substrates (e.g., metallized polyethylene terephthalate (PET), metallized polyamide, and/or metallized polypropylene), and/or coatings comprising SiOx or AlOx compounds can be used to provide low oxygen transmission to a film. In some embodiments, the barrier layer(s) can provide the film with a gas (e.g., oxygen) permeability of less than or equal to 500 cc/sqm/24 hrs/atm, in some embodiments less than 100 cc/sqm/24 hrs/atm, in some embodiments less than 50 cc/sqm/24 hrs/atm, and in some embodiments less than 25 cc/sqm/24 hrs/atm, measured in accordance with ASTM D-3985 at 23° C. and relative humidity (RH %) 100% inside (sealant layer side) and 50% outside.

The term “oxygen transmission rate” or “OTR” refers to the steady state rate at which gaseous oxygen permeates through a film or a layer at certain conditions of temperature and relative humidity (RH %). OTR is measured according to ASTM D-3985.

As used herein, the terms “seal strength”, “seal opening strength”, “seal force”, “seal opening force” and the like refer to the maximum force required to separate two sealed films. Seal strength can be measured according to ASTM F88.

As used herein, the term “seal” refers to any seal of a first region of an outer film surface to a second region of an outer film surface, formed by means of heat or any type of adhesive material, or otherwise. In some embodiments, the seal can be formed by heating the regions to at least their respective seal initiation temperatures. The sealing can be performed by any one or more of a wide variety of methods, including (but not limited to) using a heat seal technique (e.g., melt-bead sealing, thermal sealing, impulse sealing, dielectric sealing, radio frequency sealing, ultrasonic sealing, hot air, hot wire, infrared radiation).

The term “flexible” is used herein to refer to specific polymeric films as well as to the resulting containers whereby improved flexibility and/or collapsibility of the container is obtained by the use of these specific polymeric films. Flexible films can be characterized by a tensile modulus of, in some embodiment, less than 1000 MPa (145,000 PSI) and in some embodiments, less than 500 MPa (73,000 PSI), as measured according to ASTM D-882. Typically, flexible films also have a thickness below 1000 microns.

As used herein, the terms “polymer” or “(co)polymer” refers to the product of a polymerization reaction, and is inclusive of homo-polymers and co-polymers. As used herein, the term “homo-polymer” refers to a polymer resulting from the polymerization of a single type of monomer, i.e., a polymer consisting essentially of a single type of repeating unit.

As used herein, the term “copolymer” refers to a polymer resulting from the polymerization of two or more types of monomers, and includes terpolymers. As used herein, the term “glass transition temperature” or “Tg” refers to the temperature at which, when cooling a polymer from a molten state, the mechanical properties of the polymer change from those of a rubber (elastic) to those of a glass (brittle). As used herein, the glass transition temperature is the midpoint glass transition temperature measured by differential scanning calorimetry (DSC) according to ASTM D-3418.

As used herein, the phrase “ethylene/alpha olefin copolymer” or “alpha olefin copolymer” refers to heterogeneous and to homogeneous polymers such as linear low density polyethylene (LLDPE) with a density usually in the range of from 0.900 g/cc to 0.930 g/cc, linear medium density polyethylene (LMDPE) with a density usually in the range of from 0.930 g/cc to 0.945 g/cc, and very low and ultra low density polyethylene (VLDPE and ULDPE) with a density lower than 0.915 g/cc, typically in the range 0.868 to 0.915 g/cc. Unless otherwise indicated, all densities herein are measured according to ASTM D1505. In some embodiments, the term can refer to homogeneous polymers such as metallocene-catalyzed EXACT® and EXCEED® homogeneous resins obtainable from Exxon, single-site AFFINITY® resins obtainable from Dow, and TAFMER® homogeneous ethylene/alpha olefin copolymer resins obtainable from Mitsui. All these materials can include co-polymers of ethylene with one or more co-monomers selected from (C4-C10)-alpha-olefin such as butene-1, hexene-1, octene-1, etc., in which the molecules of the copolymers comprise long chains with relatively few side chain branches or cross-linked structures. As used herein, the phrase “heterogeneous polymer” or “polymer obtained by heterogeneous catalysis” refers to polymerization reaction products of relatively wide variation in molecular weight and relatively wide variation in composition distribution, i.e., typical polymers prepared, for example, using conventional Ziegler-Natta catalysts, for example, metal halides activated by an organometallic catalyst, i.e., titanium chloride, optionally containing magnesium chloride, complexed to trialkyl aluminum and may be found in patents such as U.S. Pat. No. 4,302,565 to Goeke et al. and U.S. Pat. No. 4,302,566 to Karol, et al. Heterogeneous catalyzed copolymers of ethylene and an alpha-olefin may include linear low-density polyethylene, very low-density polyethylene and ultra low-density polyethylene. Some copolymers of this type are available from, for example, The Dow Chemical Company (Dow) and sold under the trademark DOWLEX resins.

As used herein, the phrase “homogeneous polymer” or “polymer obtained by homogeneous catalysis” refers to polymerization reaction products of relatively narrow molecular weight distribution and relatively narrow composition distribution. Homogeneous polymers are structurally different from heterogeneous polymers, in that homogeneous polymers exhibit a relatively even sequencing of co-monomers within a chain, a mirroring of sequence distribution in all chains, and a similarity of length of all chains, i.e., a narrower molecular weight distribution. This term includes those homogeneous polymers prepared using metallocenes, or other single-site type catalysts, as well as those homogenous polymers that are obtained using Ziegler Natta catalysts in homogenous catalysis conditions.

The co-polymerization of ethylene and alpha-olefins under homogeneous catalysis, for example, co-polymerization with metallocene catalysis systems which include constrained geometry catalysts, i.e., monocyclopentadienyl transition-metal complexes is described in U.S. Pat. No. 5,026,798 to Canich. Homogeneous ethylene/alpha-olefin copolymers may include modified or unmodified ethylene/alpha-olefin copolymers having a long-chain branched (8-20 pendant carbons atoms), alpha-olefin comonomer available from The Dow Chemical Company, known as AFFINITY and ATTANE resins, TAFMER linear copolymers obtainable from the Mitsui Petrochemical Corporation, and modified or unmodified ethylene/alpha-olefin copolymers having a short-chain branched (3-6 pendant carbons atoms) alpha-olefin comonomer, known as EXACT resins obtainable from ExxonMobil Chemical Company.

As used herein, the term “ethylene vinyl alcohol” or “EVOH” refers to ethylene/vinyl alcohol copolymer. EVOH includes saponified or hydrolyzed ethylene/vinyl acetate copolymers, and refers to a vinyl alcohol copolymer having an ethylene comonomer, prepared by (for example) hydrolysis of vinyl acetate copolymers, or by chemical reactions with polyvinyl alcohol. In some embodiments, the degree of hydrolysis or saponification can be at least 50%, or at least 85%, or at least 90%. In some embodiments, the ethylene comonomer content can be comprised from about 25 to about 48 mole %, preferably from about 32 to about 44 mole %.

As used herein, the term “poly(glycolic acid)” or “PGA” refers to polymers comprising glycolic acid as a main component and includes copolymerized polyglycolic acids obtained by copolymerization of polyglycolic acids with other ester bond forming components, such as hydroxycarboxylic acid, lactones, dicarboxylic acid, diol, and substances obtained by mixing these polymers with additives as sub-components.

As used herein the expression “amorphous” refers to a polymer with an absence of a regular three-dimensional arrangement of molecules or subunits of molecules extending over distances, which are large relative to atomic dimensions. However, regularity of structure exists on a local scale, see “Amorphous Polymers,” in Encyclopedia of Polymer Science and Engineering, 2nd Ed., pp. 789-842 (J. Wiley & Sons, Inc. 1985). This document has a Library of Congress Catalogue Card Number of 84-19713.

“Amorphous PET” refers to a PET having no measurable melting point (less than 0.5 cal/g) by differential scanning calorimetry (DSC) or no heat of fusion as measured by DSC using for example ASTM D-3417. The term also includes polyesters that are obtained and marketed under a (semi) crystalline form but become amorphous after they are heated during extrusion. Amorphous PET has a low degree of crystallinity, typically 5 to 10%.

As used herein, the term “(co)polyester” refers to both homo- and co-polyesters, wherein homo-polyesters are defined as polymers obtained from the condensation of one dicarboxylic acid with one diol and co-polyesters are defined as polymers obtained from the condensation of at least one dicarboxylic acids with at least two different diols or at least two different dicarboxylic acids with at least one diol. Suitable polyester resins are, for instance, polyesters of ethylene glycol and terephthalic acid, or polyesters of butylene glycol and terephthalic acid. Preference is given to (co)polyesters which contain ethylene units or butylene units and include, based on the dicarboxylate units, at least 90 mol %, more preferably at least 95 mol %, of terephthalate units. The remaining monomer units are selected from other dicarboxylic acids or diols. As used herein, the term “polyethylene terephthalate”, also abbreviated “PET” refers to the homopolyester resulting from the condensation of ethylene glycol and terephthalic acid. PET includes at least 90 mol % of terephthalate units. As used herein, the term “polybutylene terephthalate”, also abbreviated “PBT” refers to the homopolyester resulting from the condensation of 1,4 butylene glycol and terephthalic acid.

When referring to co-polyesters of PET, polymers obtained by copolymerization of terephthalic acid, ethylene glycol and a further glycol and/or a further dicarboxylic acid are meant. An example of a copolyester of PET is PETG, obtained by copolymerization of terephthalic acid, ethylene glycol and a further glycol, for example cyclohexanedimethanol. Similarly, when referring to copolyesters of PBT, polymers obtained by copolymerization of terephthalic acid, 1,4 butylene glycol and a further glycol and/or a further dicarboxylic acid are meant. An example of a copolyester of PBT is PBTG, obtained by copolymerization of terephthalic acid, butylene glycol and a further glycol.

As used herein, “polypropylene”, also abbreviated “PP” includes both polypropylene homopolymers resulting from polymerization of propylene repeating units, and polypropylene copolymers, resulting from copolymerization of propylene with other repeating units, generally with ethylene. As used herein the term “polyamide” refers to high molecular weight polymers having amide linkages along the molecular chain, and refers more specifically to synthetic polyamides such as nylons. Such term encompasses both homopolyamides and co- (or ter-) polyamides. It also specifically includes aliphatic polyamides or co-polyamides, aromatic polyamides or co-polyamides, and partially aromatic polyamides or co-polyamides, modifications thereof and blends thereof. The homo-polyamides are derived from the polymerization of a single type of monomer comprising both the chemical functions which are typical of polyamides, i.e. amino and acid groups, such monomers being typically lactams or aminoacids, or from the polycondensation of two types of polyfunctional monomers, i.e. polyamines with polybasic acids. The co-, ter-, and multi-polyamides are derived from the copolymerization of precursor monomers of at least two (or three or more) different polyamides. As an example, in the preparation of the co-polyamides, two different lactams may be employed, or two types of polyamines and polyacids, or a lactam on one side and a polyamine and a polyacid on the other side. Exemplary polymers are polyamide 6, polyamide 6/9, polyamide 6/10, polyamide 6/12, polyamide 11, polyamide 12, polyamide 6/12, polyamide 6/66, polyamide 66/6/10, modifications thereof and blends thereof. Said term also includes crystalline or partially crystalline, aromatic or partially aromatic polyamides such as polyamide 6I/6T or polyamide MXD6.

As used herein, the phrase “amorphous polyamide” refers to polyamides (or “nylons”) with an absence of a regular three-dimensional arrangement of molecules or subunits of molecules extending over distances, which are large relative to atomic dimensions. However, regularity of structure exists on a local scale, see “Amorphous Polymers,” in Encyclopedia of Polymer Science and Engineering, 2nd Ed., pp. 789-842 (J. Wiley & Sons, Inc. 1985). This document has a Library of Congress Catalogue Card Number of 84-19713.

“Amorphous polyamides” includes polyamides that are lacking in crystallinity as shown by the lack of an endotherm crystalline melting peak in a Differential Scanning calorimeter (“DSC”) measurement (ASTM D-3417). More precisely, amorphous polyamides have no measurable melting point (less than 0.5 cal/g) or no heat of fusion as measured by DSC using ASTM D 3417. Such polyamides include those prepared from condensation polymerization reactions of diamines with dicarboxylic acids. For example, an aliphatic diamine is combined with an aromatic dicarboxylic acid, or an aromatic diamine is combined with an aliphatic dicarboxylic acid to give suitable amorphous polyamides.

As used herein, terms identifying polymers, such as “polyamide”, “polyester”, etc. are in general inclusive of not only polymers comprising repeating units derived from monomers known to polymerize to form a polymer of the named type, but are also inclusive of comonomers, derivatives, etc. which can copolymerize with monomers known to polymerize to produce the named polymer. For example, the term “polyamide” encompasses both polymers comprising repeating units derived from monomers, such as caprolactam, which polymerize to form a polyamide, as well as copolymers derived from the copolymerization of caprolactam with a comonomer which when polymerized alone does not result in the formation of a polyamide.

The expression “a blend of polyamides” refers to a blend of two or more polyamides, the expression “a blend of PET and/or copolymers thereof” refers to a blend of two or more PET, or two or more copolyesters of PET, or at least one PET and at least one copolyester of PET.

As used herein, the term “cyclic olefin copolymer”, or “cycloolefin copolymer”, also abbreviated “COC”, refers to copolymers of cyclic olefins (cycloolefins) and alpha-olefins, preferably ethylene-alpha-olefins. Suitable cyclic olefins for copolymerisation are monocyclic or multicyclic, preferably bicyclic cycloolefins, in which the rings preferably have 3 to 6 ring members. In multicyclic cycloolefins, preferably only one ring is a cycloolefinic ring whereas the other rings are cycloalkyl rings. The cycloolefin or cycloolefinic part of the multicycled cycloolefin is preferably a cyclo pentene or cyclohexene ring. Most preferably, the cycloolefin is norbornene. Suitable alpha-olefins for copolymerisation may have from 2 to 20 carbon atoms, but ethylene and propylene are preferred. Exemplary cyclic olefin copolymers are ethylene-norbornene copolymers marketed under the name TOPAS.

As used herein, “elastomeric cyclic olefin copolymers” refers to cyclic olefin copolymers having glass transition temperature (Tg) (measured according to ASTM D-3418) lower than 20° C. and elongation at break (measured according to ASTM D-882 at 23° C.) above 100%.

As used herein the term “ionomer” designates metal salts of acidic copolymers, such as metal salts of ethylene/acrylic acid copolymers (EAA) or metal salts of ethylene/methacrylic acid copolymers (EMAA), wherein the metal cation can be an alkali metal ion, a zinc ion or other multivalent metal ions. These resins are available, for instance, from DuPont under the trade name Surlyn™. As used herein, the term “extrusion” is used with reference to the process of forming continuous shapes by forcing a molten plastic material through a die, followed by cooling (quenching) or chemical hardening. Immediately prior to extrusion through the die, the relatively high-viscosity polymeric material may be fed into a rotating screw of variable pitch, i.e., an extruder, which forces the polymeric material through the die.

As used herein, the term “coextrusion” refers to the process of extruding two or more materials through a single die with two or more orifices arranged so that the extrudates merge and weld together into a laminar structure before chilling, i.e., quenching. The term “coextrusion” as used herein also includes “extrusion coating”.

As used herein, the term “extrusion coating” refers to processes by which a “coating” of molten polymer(s), comprising one or more layers, is extruded onto a solid “substrate film” in order to coat the substrate film with the molten polymer coating to bond the substrate and the coating together, thus obtaining a complete film.

As used herein, the term “inert” or “biologically inert” refers to a property of a material (e.g. film or surface thereof) whereby the material does not chemically react or interfere with the biological material or media inside the bioreactor and/or does not scalp or leach into biological material or media.

As used herein, the term “migratory surface additive” or “migratory surface agent” refers to an additive or agent present in one or more of the layers of the film, which is capable of moving through the film layer(s) and blooming to the film surface(s). On the contrary, “not-migratory” additives or agents are those which cannot bloom to the film surface, i.e. whose concentration at the surface does not change with time after crystallization (solidification after melt state) of the film.

The term “bioprocessing” as used herein refers to any process that uses living cells, or their components, or their aggregates (e.g., bacteria, enzymes, chloroplasts, tissues, and the like). For example, in some embodiments, bioprocessing can include processes for the production of a product by growing cultures of cells or microorganisms, processes of growing cultures of cells or microorganisms, and/or processes for the bioconversion of one material to another.

The term “container” as used herein includes, but is not limited to, any of a wide variety of packages or storage devices including pouches, bags, boxes, cartons, envelopes, bottles, and the like manufactured from a polymeric film. The term “container” also includes any packaging or storage device that has been designed for or in support of bioprocessing applications.

The terms “bioprocessing container”, “container for bioprocessing applications”, or “bioreactor” as used herein refer to a container suitable for use in bioprocessing applications (such as, but not limited to, growing cell cultures). Alternatively, or in addition, bioprocessing containers can be used to house any of a wide variety of biological fluids such as serum, buffers, ultrapure water, blood, physiological solutions, and the like.

The term “cell” as used herein refers to any cellular matter that can be maintained in a bioprocessing container. For example, in some embodiments, the term “cell” can comprise (but is not limited to) eukaryotic cells (such as yeast cells, insect cells, mammalian cells, plant cells). In other embodiments, the term “cell” can comprise prokariotic cells (typically bacterial cells). It should be understood that the term “cell” can also encompass any of a wide variety of cellular components.

The term “inoculating” or “inoculation” as used herein refers to the introduction of at least one biological component (such as, for example, a cell) into a medium to begin a culture.

The term “liquid medium” as used herein includes any liquid medium that can be used for conventional methods of bioprocessing, such as (but not limited to) cell culture medium.

The term “internal surface”, when used in reference to a container, for example (but not limited to) a bioprocessing container, refers to the surface of the container which is directed towards the inside of the container and is contact with the material (e.g. the liquid medium) which is housed in the container. The term “external surface”, referred to a container, refers to the surface of the container which is directed towards the environment and is not in contact with the material housed in the container. In embodiments, the internal surface of a container is defined by the sealant layer of a polymeric film, while the external surface of a container is defined by the skin (or abuse) layer of a film. All compositional percentages used herein are percentages by weight unless otherwise stated.

### III. The Presently Disclosed Film

### III.A. Generally

The presently disclosed subject matter is directed to a polymeric, multilayer film suitable for use in a wide variety of applications, such as (but not limited to) the manufacture of bioprocessing containers (bioreactors).

Specifically, the presently disclosed polymeric film comprises:
a sealant layer comprising:
(i) 58-95.5 wt % in respect of the total weight of the sealant layer of one or more ethylene/alpha olefin copolymers having an average density lower than 0.922 g/cc;
(ii) 4-10 wt % in respect of the total weight of the sealant layer of one or more polymers having an average density higher than or equal to 0.950 g/cc and selected among one or more ethylene/alpha olefin copolymers and/or one or more ethylene homopolymers;
(iii) 0.5-2.0 wt % in respect of the total weight of the sealant layer of high molecular weight siloxane polymers;
(iv) optionally, 5-30 wt % in respect of the total weight of the sealant layer of elastomeric cyclic olefin copolymers (COC) with glass transition temperature (Tg) lower than 20° C.;
optionally, at least one gas barrier layer comprising one or more polymers selected from the group consisting of poly(glycolic acid) (PGA), polyamides, ethylene vinyl alcohol (EVOH) and mixtures thereof;
an outer skin layer comprising one or more polymers selected from the group consisting of: (co)polyesters, polyamides, high-density polyethylene (HDPE), polypropylene (PP) and their admixtures.

The disclosed film comprises a sealant layer whose polymeric composition grants it an improved seal strength even when the film undergoes high temperatures (e.g. higher than 60° C.) and is substantially free from migratory surface additives and from metal containing particulate additives. Accordingly, a bioprocessing container manufactured with the disclosed film has a biologically inert internal surface (i.e. surface towards the hosted material) which does not inhibit the growth of biological cell cultures and/or does not interfere with the composition and the stability of the biological fluids stored therein.

In embodiments, the disclosed film may further comprise at least one gas barrier layer which ensures that the film maintains gas barrier properties.

The disclosed film also comprises an outer skin layer comprising polymers with high melting temperatures (higher than the melting temperatures of the sealant layer) to allow for a wide range of seal temperatures when the film is sealed to form a container. The polymers of the outer skin layer also offer toughness and abuse resistance to the film and the containers manufactured with it.

In some embodiments, the disclosed film may comprise from 3 to 20 layers; in some embodiments, from 3 to 15 layers; in some embodiments, from 4 to 12 layers. Accordingly, the disclosed film may comprise 3, 4, 5, 6, 7, 8, 9, 10, 11, 12, 13, 14, 15, 16, 17, 18, 19, or 20 layers. In an embodiment, the disclosed film may comprise 6 layers. In another embodiment, the disclosed film may comprise 10 layers. In another embodiment, the disclosed film may comprise 11 layers. A person skilled in the art will also recognize that the disclosed film may comprise more than 20 layers, such as in embodiments wherein the film components comprise microlayering technology.

The disclosed film can be manufactured using any suitable process known in the art, including (but not limited to) coextrusion, extrusion coating, lamination and combinations thereof. Suitable processes for manufacturing the disclosed film are described for example in U.S. Pat. No. 6,769,227 in the name of Mumpower, U.S. Pat. Nos. 3,741,253 and 4,278,738 in the name of Brax et al., U.S. Pat. No. 4,284,458 in the name of Schirmer, and U.S. Pat. No. 4,551,380 in the name of Schoenberg, each of which is hereby incorporated by reference in its entirety.

The disclosed film can have any total thickness desired, so long as the film provides the desired optical and mechanical properties for the particular packaging operation or use in which the film is employed, e.g., clarity, modulus, seal strength, and the like. Final thickness can vary, depending on manufacturing process, end use application, and the like. Typical thicknesses can range from 30 to 600 microns; in some embodiments, from 50 to 508 microns; in some embodiments, from 80 to 450 microns; in some embodiments, from 100 to 400 microns; in some embodiments, from 150 to 380 microns, from 200 to 360 microns; from 260 to 340 microns, from 280 to 320 microns. In some embodiments, the disclosed films can have a total thickness of about 305 microns or of about 315 microns. A person skilled in the art would also recognize that the presently disclosed subject matter also includes embodiments wherein film thicknesses lie outside the ranges set forth herein. In some embodiments, the disclosed film can comprise printed product information such as (but not limited to) product size, type, name of manufacturer, instructions for use, and the like. Such printing product information and the methods to apply them onto the film are well known to those skilled in the field of polymeric films.

In some embodiments, the disclosed film is biologically inert, i.e., its sealant layer, which forms the internal, material-contacting surface of a container made with the film is compatible and does not interfere with cell culture and/or with the biological material housed therein. In particular, the disclosed film is substantially free from migratory surface additives and from metal containing particulate additives. In some embodiments, the term “substantially free” refers to a total amount of migratory surface additives and/or metal containing particulate additives of 0.05 wt % or less, based on the total weight of the film. Migratory surface additives are well known to people skilled in the art and can include (but are not limited to) antifogging agents, antistatic agents, migratory amide waxes working as slip agents, protein coatings, therapeutic agent coatings, binding agents, and the like.

### III.B. Sealant Layer

The disclosed multilayer film comprises a sealant layer, which comprises:
(i) 58-95.5 wt % in respect of the total weight of the sealant layer of one or more ethylene/alpha olefin copolymers having an average density lower than 0.922 g/cc;
(ii) 4-10 wt % in respect of the total weight of the sealant layer of one or more polymers having an average density higher than or equal to 0.950 g/cc and selected among one or more ethylene/alpha olefin copolymers and/or one or more ethylene homopolymers; and
(iii) 0.5-2.0 wt % in respect of the total weight of the sealant layer of high molecular weight siloxane polymers.

The inventors have surprisingly found that mixing ethylene/alpha olefin copolymers and/or ethylene homopolymers with an average density ≥0.950 g/cc and high molecular weight siloxane polymers with ethylene/alpha olefin copolymers with an average density <0.922 g/cc in the stated amounts, a sealant layer is obtained which does not tend to stick on itself when the film is flattened after being manufactured as a tube through round technologies, i.e. does not show blocking effect. This composition of the sealing layer allows to avoid incorporating olefin hydrocarbon polymers with Tg≥25° C. (such as COC with Tg≥25° C.) which, while reducing the sticking (blocking) effect of the film, proved to reduce the seal strength, as discussed in the background section. In other words, even if the polymers with an average density ≥0.950 g/cc (ethylene/alpha olefin copolymers and/or ethylene homopolymers) have Tg below 20° C., when mixed with ethylene/alpha olefin copolymers with an average density <0.922 g/cc in the stated amounts are capable of reducing the blocking effect. The high molecular weight siloxane polymers also contribute to reducing the sticking of the film, thus allowing to obtain a film with negligible blocking effect.

At the same time, the polymers with an average density ≥0.950 g/cc (ethylene/alpha olefin copolymers and/or ethylene homopolymers) do not negatively affect the seal strength, even when the film is exposed to temperatures higher than 60° C.

In some embodiments, the ethylene/alpha olefin copolymers with an average density <0.922 g/cc can include heterogeneous polymers such as linear low density polyethylene (LLDPE) with a density usually in the range of from 0.900 g/cc to 0.930 g/cc, and very low and ultra low density polyethylene (VLDPE and ULDPE) with a density lower than 0.915 g/cc. These ethylene/alpha olefin copolymers are selected and mixed in a proper way so that the resulting average density is <0.922 g/cc. In some embodiments, the resulting average density can be below 0.918 g/cc or below 0.914 g/cc. In some embodiments, the resulting average density can be about 0.910 g/cc.

In some embodiments, the ethylene/alpha olefin copolymers with an average density below 0.922 g/cc can include homogeneous polymers, such as metallocene-catalyzed EXACT® and EXCEED® homogeneous resins obtainable from Exxon, single-site AFFINITY® resins obtainable from Dow, and TAFMER® homogeneous ethylene/alpha olefin copolymer resins obtainable from Mitsui. All these materials generally include co-polymers of ethylene with one or more co-monomers selected from (C3-C10)-alpha-olefin such as propene-1, butene-1, hexene-1, octene-1, etc., in which the molecules of the copolymers include long chains with relatively few side chain branches or crosslinked structures.

In some embodiments, the ethylene/alpha olefin copolymers with an average density below 0.922 g/cc can be selected among LLDPE, VLDPE, and blends thereof.

Suitable ethylene/alpha olefin copolymers with an average density below 0.922 g/cc are for example CV77539 commercialized by Westlake Chemical and EXACT 3024 commercialized by ExxonMobil.

Generally, the sealant layer may comprise 58-95.5 wt % of one or more ethylene/alpha olefin copolymers with an average density <0.922 g/cc. In some embodiments, the sealant layer may comprise 65-95.5 wt % of such ethylene/alpha olefin copolymers. In some embodiments, the sealant layer may comprise 75-95.5 wt % of such ethylene/alpha olefin copolymers.

In some embodiments, the polymers with an average density ≥0.950 g/cc can be selected among high density polyethylene (HDPE) with a density usually in the range of from 0.930 g/cc to 0.970 g/cc, medium density polyethylene (MDPE) with a density usually in the range of from 0.926 g/cc to 0.940 g/cc and mixtures thereof. These polymers (ethylene/alpha olefin copolymers and/or ethylene homopolymers) are selected and mixed in a proper way so that the resulting average density is higher than or equal to 0.950 g/cc.

In some embodiments, the polymer with an average density ≥0.950 g/cc can be HDPE. A suitable HDPE resin can be for example SCLAIR 2908 commercialized by Nova Chemicals.

The sealant layer also comprises 0.5 to 2.0 wt % in respect of the total weight of the sealant layer of high molecular weight siloxane polymers.

High molecular weight siloxane polymers are known in the field of polymeric films as not-migratory slip additives, which help reducing the friction of the film on the manufacturing and converting equipment (e.g on rolls) thus assisting film processing and conversion. In the disclosed films, the high molecular weight siloxane polymers, used together with the polymers with an average density ≥0.950 g/cc (ethylene/alpha olefin copolymers and/or ethylene homopolymers), surprisingly proved capable of contributing to reduce the blocking effect. Generally speaking, since polymers with an average density ≥0.950 g/cc and high molecular weight siloxane polymers cooperate in reducing the blocking of the film, when polymers with an average density ≥0.950 g/cc are present in high amounts (within their disclosed range), the high molecular weight siloxane polymers may be present in low amounts (within their disclosed range), and vice versa.

High molecular weight siloxane polymers are generally mixed with a polyolefin carrier, typically LLDPE, VLDPE, or LDPE (low density polyethylene) to form a masterbatch, as it is known in the art.

Polydimethylsiloxanes are typically used as high molecular weight siloxane polymers. Examples of siloxane-comprising masterbatches to be used in the sealant layer of the present film are MB50-802 and MB25-235, both by Dupont, comprising polydimethylsiloxane in LDPE carrier.

In some embodiments, the sealant layer may also optionally comprise 5-30 wt % in respect of the total weight of the sealant layer of elastomeric cyclic olefin copolymers (COC) with glass transition temperature (Tg) lower than 20° C. COC are known for their inertness which might help cell culturing and harvesting and do not interfere with the bioprocessing materials. Specifically, elastomeric COC with Tg below 20° C. can increase toughness, flexibility and pinhole resistance thus making the internal surface of a bioprocessing container made with the disclosed film more resistant to injuries coming from friction, folding, piercing.

An example of elastomeric COC suitable for use in the sealant layer of the presently disclosed film is Topas E-140 from Topas Advanced Polymers, Inc. In some embodiments, the sealant layer may comprise 75-95.2 wt % of one or more ethylene/alpha olefin copolymers with an average density <0.922 g/cc, 4-6 wt % of one or more polymers with an average density ≥0.950 g/cc selected among ethylene/alpha olefin copolymers and/or ethylene homopolymers and 0.8 to 2.0 wt % of high molecular weight siloxane polymers. The polymer with an average density ≥0.950 g/cc is preferably HDPE.

In other embodiments, the sealant layer may comprise 75-93.5 wt % of one or more ethylene/alpha olefin copolymers with an average density <0.922 g/cc, 6-10 wt % of one or more polymers with an average density ≥0.950 g/cc selected among ethylene/alpha olefin copolymers and/or ethylene homopolymers and 0.5 to 1.2 wt % of high molecular weight siloxane polymers. The polymer with an average density ≥0.950 g/cc is preferably HDPE.

### III.C. Barrier Layer(s)

In some embodiments, the disclosed film may include at least one gas barrier layer comprising one or more polymers selected from the group consisting of poly(glycolic acid) (PGA), polyamides, ethylene vinyl alcohol (EVOH) and mixtures thereof.

PGA is a known polymeric material and can be prepared using any of a wide variety of methods known in the art. For example, PGA can be prepared using the ring-opening polymerization of glycolide using stannous octoate catalyst (D. K. Gilding and A. M. Reed in Polymer, Vol. 20, p. 1459 (1979)). Alternatively, PGA can be produced by a process such as dehydration polycondensation of glycolic acid, dealcoholization polycondensation of an alkyl glycolate, desalting polycondensation of a glycolic acid salt, and/or ring-opening polymerization of glycolide.

Polyamides are known in the art and include polymers having amide linkages (such as synthetic polyamides) which can be either aliphatic or aromatic and either in semi-crystalline or amorphous form. Suitable polyamides used in the barrier layer(s) of the disclosed film can include both homo-polyamides and co- (or ter-)polyamides.

For example, polyamides used in the barrier layer(s) can include (but are not limited to) nylon homopolymers and copolymers such as those selected from the group comprising: nylon 4,6 (poly(tetramethylene adipamide)), nylon 6 (polycaprolactam), nylon 6,6 (poly(hexamethylene adipamide)), nylon 6,9 (poly(hexamethylene nonanediamide)), nylon 6,10 (poly(hexamethylene sebacamide)), nylon 6,12 (poly(hexamethylene dodecanediamide)), nylon 6/12 (poly(caprolactam-co-laurallactam)), nylon 6,6/6 (poly(hexamethylene adipamide-co-caprolactam)), nylon 6/66 (poly(caprolactam-co-hexamethylene adipamide)), nylon 66/610 (e.g., manufactured by the condensation of mixtures of nylon 66 salts and nylon 610 salts), nylon 6/69 resins (e.g., manufactured by the condensation of epsilon-caprolactam, hexamethylenediamine and azelaic acid), nylon 11 (polyundecanolactam), nylon 12 (polyauryllactam), nylon MXD6, nylon MXDI, nylon 6I/6T, and copolymers or mixtures thereof.

In some embodiments, the barrier layer(s) may comprise MXD6.

In some embodiments, the barrier layer(s) may comprise one or more amorphous polyamides.

EVOH is a copolymer of ethylene and vinyl alcohol repeating units and can contain small amounts of other monomer units, such as vinyl ester units. EVOH can be prepared by saponification, partial alcoholysis of ethylene-vinyl ester copolymers, and/or complete alcoholysis of ethylene-vinyl ester copolymers. The molar proportion of ethylene in an EVOH copolymer to be used in the barrier layer(s) can range from 3 mol % to 75 mol %; typical molar proportion of ethylene in EVOH are from 10 mol % to 50 mol %, from 20 mol % to 52 mol %, from 23 mol % to 48 mol %.

The barrier layer(s) can comprise two or more EVOH copolymers (i.e. an EVOH blend) with different molar proportions of ethylene.

In some embodiments, the barrier layer(s) can comprise a major proportion of one or more polymers selected from the group consisting of PGA, polyamides, EVOH and mixtures thereof. For example, the barrier layer(s) can comprise at least 60%, at least 70%, at least 80%, at least 90%, at least 95% by weight with respect to the weight of the barrier layer, of one or more polymers selected from the group consisting of PGA, polyamides, EVOH and mixtures thereof. In an embodiment, the barrier layer(s) can consist of one or more polymers selected from the group consisting of PGA, polyamides, EVOH and mixtures thereof.

In an embodiment, the film may have only one barrier layer. Such only one barrier layer may comprise a major proportion, preferably at least 60%, at least 70%, at least 80%, at least 90%, at least 95% by weight with respect to the weight of the barrier layer of one or more polymers selected from the group consisting of PGA, polyamides, EVOH and mixtures thereof.

In an embodiment, such only one barrier layer may consist of one or more polymers selected from the group consisting of PGA, polyamides. EVOH and mixtures thereof.

In an embodiment, such only one barrier layer may consist of EVOH.

Suitable EVOH resins are for example EVASIN EV3251F and EVASIN EV3851F marketed by Chang Chun Petrochemicals Ltd., SOARNOL ET3803 by Mitsubishi Chemical Corporation, Eval L171B by EVALCA/Kuraray.

In an embodiment, such only one barrier layer may consist of a PGA/polyamide blend, consisting of up to about 50 wt % PGA and at least about 50 wt % polyamide, based on the total weight of the barrier layer.

In an embodiment, the film may have two or more barrier layers. In an embodiment, such two or more barrier layers may independently comprise a major proportion, preferably at least 60%, at least 70%, at least 80%, at least 90%, at least 95% by weight with respect to the weight of the barrier layer of one or more polymers selected from the group consisting of PGA, polyamides. EVOH and mixtures thereof.

In an embodiment, such two or more barrier layers may independently consist of one or more polymers selected from the group consisting of PGA, polyamides, EVOH and mixtures thereof.

In an embodiment, such two or more barrier layers may both consist of EVOH.

In some embodiments, the disclosed film may comprise a first barrier layer positioned adjacent to the sealant layer and a second barrier layer positioned adjacent to the skin layer. The first barrier layer may comprise a major proportion of one or more polymers selected from the group consisting of PGA, polyamides, EVOH and mixtures thereof and the second barrier layer may comprise a major proportion of EVOH. Such embodiments are disclosed in U.S. Pat. No. 8,722,164. A first and second barrier layer according to these embodiments ensure that the film maintains gas barrier properties under a wide variety of conditions, i.e. under low, intermediate and high relative humidity (RH %). In fact, as it is well known, the barrier properties of EVOH are suitable in low humidity conditions, but degrade substantially when exposed to high humidity. With the term “high relative humidity” reference is made to a relative humidity ranging from 70% and 100%, preferably from 75% and 100%, from 80% and 100%, from 85% and 100%.

With the term “low relative humidity” reference is made to a relative humidity ranging from 0% and 30%, preferably from 0% and 20%, from 0% and 10%, from 0% and 5%.

A film having such first and second barrier layer may exhibit an oxygen transmission rate (OTR) after at least one hour in high, intermediate, or low relative humidity conditions ranging, in some embodiments, from 0 to 500 cc/sqm/atm/day, in some embodiments, from 0 to 300 cc/sqm/atm/day, and in some embodiments, from 0 to 200 cc/sqm/atm/day.

In another embodiment, the film may not have any barrier layers.

### III.D. Skin Layer

The disclosed film comprises an outer skin layer comprising one or more polymers selected from the group consisting of: (co)polyesters, polyamides, high-density polyethylene (HDPE), polypropylene (PP) and their admixtures.

In some embodiments, the skin layer can comprise a major proportion of one or more polymers selected from the group consisting of (co)polyesters, polyamides, HDPE, PP and their mixtures. For example, the skin layer can comprise at least 60%, at least 70%, at least 80%, at least 90%, at least 95% by weight with respect to the weight of the skin layer, of one or more polymers selected from the group consisting of (co)polyesters, polyamides, HDPE, PP and their mixtures.

In an embodiment, the skin layer can consist of one or more polymers selected from the group consisting of (co)polyesters, polyamides, HDPE, PP and their mixtures.

In an embodiment, the skin layer can comprise a major proportion, at least 60%, at least 70%, at least 80%, at least 90%, at least 95% by weight with respect to the weight of the skin layer, of one or more polymers selected from the group consisting of (co)polyesters and polyamides.

In an embodiment, the skin layer can consist of one or more polymers selected from the group consisting of (co)polyesters and polyamides.

In an embodiment, polyesters can be selected among PET, PBT and mixtures thereof.

In an embodiment, co-polyesters can be selected among copolymers of PET, copolymers of PBT and mixtures thereof.

A suitable example of (co)polyesters is Ecdel 9965 marketed by Eastman Chemical.

In an embodiment, the skin layer can comprise a major proportion, at least 60%, at least 70%, at least 80%, at least 90%, at least 95% by weight with respect to the weight of the skin layer, of a blend of (co)polyesters, for example a blend of PET and/or copolymers thereof, optionally wherein at least one PET and/or copolymers thereof is amorphous and has a Tg (measured in accordance with ASTM D-3418) higher than or equal to 50° C.

In an embodiment, the skin layer can comprise a major proportion, at least 60%, at least 70%, at least 80%, at least 90%, at least 95% by weight with respect to the weight of the skin layer, of a polyamide or a blend of polyamides, optionally wherein at least one polyamide of the blend is amorphous and has a Tg (measured in accordance with ASTM D-3418) higher than or equal to 50° C. In an embodiment, the skin layer can consist of such polyamide or blend of polyamides.

Suitable examples of polyamides are elastomeric polyamides such as Vestamid E40-53, Vestamid L1670, Vestamid D16 (marketed by Evonik Corp.), Grilamid ELY20NZ, Grilamid 2702, Grilamid 2475, and Grilamid 60 (marketed by EMS Grivory).

In an embodiment, the skin layer can comprise a major proportion, at least 60%, at least 70%, at least 80%, at least 90%, at least 95% by weight with respect to the weight of the skin layer of one or more (co)polyesters selected among PBT and copolymers thereof, for example PBTG.

In an embodiment, the skin layer can consist of one or more (co)polyesters selected among PBT and copolymers thereof, for example PBTG.

In an embodiment, the skin layer can comprise a major proportion, at least 60%, at least 70%, at least 80%, at least 90%, at least 95% by weight with respect to the weight of the skin layer, of PBTG. In an embodiment, the skin layer can consist of PBTG.

Suitable examples of PBTG are ARNITEL VT3104 and ARNITEL EM400, both marketed by DSM, Hytrel 4068 and Hytrel 4039 marketed by Dupont.

The polymers used in the skin layer of the disclosed film have high melting temperatures (typically higher than 180° C.) and however higher than the melting temperature of the sealant layer, in order to allow for a wide range of seal bar temperatures when the film is to be sealed for manufacturing the container. In addition, the polymers of the skin layer suitably belong to the polymers family known in the art to be “thermoplastic elastomers”. Such polymers typically have a tensile modulus of less than 500 MPa and an elongation at break higher than 350% (measured according to ASTM D-882 at 23° C.), further to a high melting temperature. These properties allow to obtain a film which is capable of withstanding deformation without pinholes generation and which does not adhere to the seal bar.

### III.E. Additional Layers

The disclosed film can comprise one or more inner “bulk” layer(s) or “structural” layer(s), as it is known to the person skilled in the art.

These layers generally comprise polymers used to improve the resistance of the film to abuse, abrasion, puncture or other potential causes of reduction of the integrity or of the appearance of the disclosed film. Bulk layers may be used also just to provide the desired thickness.

Polymers suitable for these layers are typically ethylene homo- and copolymers, e.g. low density polyethylene (LDPE), ethylene-vinyl acetate copolymers (EVA), linear low density polyethylenes (LLDPE), linear very low density polyethylenes (VLDPE), ethylene/acrylic acid copolymers (EAA), ethylene/methacrylic acid copolymers (EMAA), ethylene/methyl acrylate copolymers (EMA), ethylene/butyl acrylate copolymers (EBA), ionomers, and mixtures thereof.

Suitable examples of VLDPE may be EXACT 3024 marketed by ExxonMobil and XUS 61520. 15L marketed by Dow.

Suitable examples of LLDPE may be CV77525 marketed by Westlake Chemical and DOWLEX 2045.03 marketed by Dow.

A suitable ethylene/butyl acrylate copolymer may be for example EMAC SP2202 marketed by Westlake Chemical.

A suitable ethylene/methyl acrylate copolymer may be for example EMAC+ SP1330 marketed by Westlake Chemical.

In some embodiments, the presently disclosed film can comprise at least one bulk layer, typically two or three bulk layers are present in the film.

The disclosed film can comprise one or more inner tie layers which improve the adherence of one layer to another layer, as it is known to those skilled in the art.

In an embodiment, the film may include tie layer(s) directly adjacent to one or both sides of the barrier layer(s), if present, to better adhere the barrier layer(s) to the adjacent bulk layer(s), if present. In an embodiment, a tie layer may also be used to better adhere the bulk layer to the outer skin layer.

Tie layers may include polymers having grafted polar groups so that the polymer is capable of covalently bonding to polar polymers such as EVOH, polyesters or polyamides.

Suitable polymers for tie layers include one or more thermoplastic polymers such as ethylene-unsaturated acid copolymers, ethylene-unsaturated ester copolymers, polyurethane, ethylene-vinyl acetate copolymers, ethylene-(meth)acrylic acid copolymers, ethylene/methyl acrylate copolymers, ethylene homo-polymers or co-polymers modified with anhydride or carboxylic acid functionalities, mixtures of these resins or mixtures of any of the above resins with an ethylene homo- or co-polymer, and the like known resins, such as, for example, anhydride modified grafted linear low density polyethylene, anhydride grafted low density polyethylene, homogeneous ethylene/alpha-olefin copolymer, anhydride grafted ethylene/vinyl acetate copolymer, anhydride grafted ethylene/methyl acrylate copolymers, anhydride grafted styrene/ethylene/butadiene/styrene copolymers.

Some suitable commercial tie resins are for example those marketed by Dow under the tradename BYNEL, e.g. BYNEL 4164, BYNEL 21E810, BYNEL 21E78, BYNEL 46E1060, BYNEL 21E787.

Various combinations of layers can be used to form multilayer film in accordance with the presently disclosed subject matter. For example, the layers sequence of the disclosed films can be selected among the following non exhaustive list, wherein A represents the sealant layer, B represents a barrier layer, C represents the outer skin layer, D represents a bulk layer and E represents a tie layer: A/B/C, A/D/B/C, A/B/D/C, A/D/B/D/C, A/D/D/B/C, A/D/B/D/D/C, A/B/D/B/C, A/D/B/D/B/D/C, A/D/B/D/B/D/D/C, A/D/D/B/D/B/D/C, A/D/E/B/E/D/C, A/D/E/B/D/C, A/D/B/E/D/C, A/D/E/B/E/D, A/D/E/B/E/B/E/D/E/C, A/D/D/E/B/E/B/E/D/E/C, A/C, A/D/C, A/E/D/E/C, A/E/C, A/D/E/D/C, A/D/E/D/E/D/C, A/E/D/E/D/E/C.

Where the multilayer film representation above includes the same letter more than once, each occurrence of the letter may represent the same composition or a different composition within the class that performs a similar function.

One or more of any of the layers of the disclosed multilayer film may include appropriate amounts of additives commonly used in the art for the desired effect. For example, such additives can include (but are not limited to) thermal stabilizers, processing aids, not-migratory slip agents, antiblock agents, antioxidants, fillers, dyes, pigments, radiation stabilizers, oxygen scavengers, antistatic agents, and the like. Advantageously, such additives should be not-migratory additives, to allow inertness of the surface of the sealant layer of the film.

In some embodiments, the amount of additives present in the film, especially in the sealant layer, is minimized such that the film or layer properties are not affected.

### IV. Uses of the Disclosed Film: Bioprocessing Container and Bioprocessing Methods

While the disclosed films can have applications in a wide variety of areas, they are particularly suitable for use in manufacturing flexible containers for bioprocessing applications. Bioprocessing containers provide a disposable environment for bioprocessing applications such as culturing cells, cell aggregates, particles, tissues, and the like. Bioprocessing containers can be stand-alone or can be used together with a wide variety of support devices to form a container-support assembly. Support devices can be, for example, bioreactors, stirred tank reactors, and the like, made of e.g. metal, glass, or hard plastic. The flexible bioprocessing container can be inserted within the support device, such that the bioprocessing occurs in the container, the support device only having a structural support role, as described in more detail below. Accordingly, a flexible bioprocessing container is disclosed, comprising at least a film as presently disclosed, wherein the film is sealed to define an interior compartment of the container and wherein the sealant layer of the disclosed film forms the internal surface of the container, i.e. the surface coming into contact with the material housed in the container. Specifically, the film is sealed on its edges to form the sidewalls of the container, which define an interior compartment.

A suitable bioprocessing container can comprise a first and a second film, sealed along their edges to form the sidewalls of the container which define an interior compartment. At least one of said first and second film are the disclosed film. Preferably, both the first and the second film are the disclosed films. In some embodiments, the disclosed film can be used to manufacture the bioprocessing container either by itself or together with another barrier or non-barrier film, thus forming a double walled container comprising two films sealed together around the container perimeter, an inner film, towards the housed material and an outer film, towards the external environment.

In these embodiments, preferably, the disclosed film is the inner film of the bioprocessing container. Double walled bioprocessing containers are popular in bioprocessing applications due to the increase in abuse resistance properties and are well known to those skilled in the art.

In some embodiments, the sealant layer of the film (which will form the internal surface of the bioprocessing container) can be treated by any of a wide variety of methods known in the art, including (but not limited to) plasma discharge, corona discharge, gas plasma discharge, ion bombardment, ionizing radiation, and/or high intensity UV light. These treatments can help to make the surface even more inert and thus suitable for cell growth.

In some embodiments, the disclosed bioprocessing container can be pre-sterilized prior to the introduction of biological materials (e.g., cells), as most cell growth procedures are carried out under aseptic conditions. For example, the disclosed containers can be sterilized by exposure to gamma radiation, ultraviolet radiation, ethylene oxide, or combinations thereof, as it is known to those skilled in the art. After the bioprocessing container has been sterilized, an appropriate liquid medium can be housed into the interior compartment of the container, depending on the particular use desired. For example, a cell culture medium can be deposited into the interior compartment of the container to grow a cell culture. The bioprocessing container can then be inoculated and incubated according to the specific cell culture conditions, as it is known in the art.

Alternatively, the cell culture medium can be pre-inoculated with cells and the pre-inoculated cell culture medium can be deposited into the interior compartment of the container. Then, the bioprocessing container can be incubated according to the specific cell culture conditions.

Thus, it is herein disclosed a method for culturing cells comprising either the steps of:
(i) providing the disclosed bioprocessing container;
(ii) introducing a liquid medium into the interior compartment of the container;
(iii) inoculating the liquid medium with cells, and
(iv) incubating the cells within the interior compartment of the container under suitable conditions for cell growth;
or the steps of:
(i) providing the disclosed bioprocessing container;
(ii) pre-inoculating a liquid medium with cells;
(iii) introducing the pre-inoculated liquid medium into the interior compartment of the container, and
(iv) incubating the cells within the interior compartment of the container under suitable conditions for cell growth.

The disclosed bioprocessing container can be configured such that the material housed in its interior compartment remains substantially in contact only with the container during use. In such embodiments, the bioprocessing container can be disposable and used for a single reaction or a single series of reactions, after which the container is discarded. Since in these embodiments the material in the bioprocessing container does not come into contact with a support device (if used), the support device can be reused without cleaning. That is, after a reaction or a series of reactions takes place in the bioprocessing container, this can be removed from the support device, disposed, and replaced by a second (e.g., disposable) bioprocessing container. A second reaction or series of reactions can be carried out in the second container without cleaning/sterilizing the reusable support device.

The disclosed bioprocessing container can include at least one access port whereby e.g. cells and/or cell culture media can be introduced and/or removed. In some embodiments, the access port can transverse both sides of the container and can be fused to the container sidewall. In some embodiments, the access port can include an internal gasket and an external gasket to ensure that there is no leakage around the port where it protrudes through the container.

In some embodiments, a syringe or other transport device can be used to introduce materials into the interior compartment of the container through the access port. It should be appreciated that any number of access ports can be provided in accordance with the disclosed bioprocessing container. For example, the bioprocessing container can have an access port that functions as an inlet for the introduction of materials into the interior compartment of the container and a separate access port that functions as an outlet. The access port(s) can be equipped with suitable measures for sealing against leakage, such as gaskets, valves and the like, as it is well known in the art.

In some embodiments, the disclosed bioprocessing container can include one or more gas removal ports. In some embodiments, the gas removal port can transverse both sides of the container and can be fused to the container sidewall. In some embodiments, the gas removal port can include an internal gasket and an external gasket to ensure that there is no leakage around the port where it protrudes through the container. As would be appreciated by those skilled in the art, the access port can function as the gas removal port in some embodiments.

In some embodiments, the disclosed bioprocessing container can comprise one or more sampling ports that can be used for sampling, analyzing (e.g., determining pH and/or amount of dissolved gases in the liquid), or for other purposes. The sampling ports can be aligned with the one or more access ports of the container. In some embodiments, sampling and/or analyzing can be accomplished through the access port, so the access port can function as the sampling port.

In some embodiments, the bioprocessing container can include a mixing system, such as a pulsating disk, paddle mixer, rocking platform, impeller, and the like. For example, the flexible bioprocessing container (optionally the whole container-support assembly) can be rotated about one axis (such as, for example, the longitudinal axis) of the container. However, the bioprocessing container and/or the whole container-support assembly can be tilted and rotated at an angle from the longitudinal axis of the container. Alternatively, or in addition, in some embodiments, the disclosed container can include a mixing system (such as an impeller) positioned within the interior compartment of the container. The impeller can be rotated using a motor that can be external or internal to the container.

In some embodiments, the disclosed bioprocessing container can include a heater, such as (but not limited to) a heating pad, a steam jacket, a circulating fluid heater, and/or a water heater. The heater can be located between the bioprocessing container and a support device, or can be incorporated into the support device or into the bioprocessing container itself. In some embodiments, the bioprocessing container can be placed inside an incubator to maintain a desired temperature.

The disclosed flexible bioprocessing containers can have any of a wide variety of sizes and shapes known in the art. In embodiments, the size and shape of the bioprocessing container can be determined by the size and shape of the support device (if any) to be used. To this end, the height and/or width of the bioprocessing container can be scaled to any desired and suitable size depending on the particular use. For example, the bioprocessing container can have a volume of 1-40, 40-100, 100-200, 200-300, 300-500, 500-750, 750-1000, 1000-2000, 2000-5000, or 5000-10000 liters. Thus, the bioprocessing container can have a volume greater than 1, 10, 20, 40, 100, 200, 500, or 1,000 liters. Volumes less than 1 liter and greater than 10,000 liters are also possible. The disclosed container is suitable for any of a wide variety of bioprocessing applications including (but not limited to) cell culturing of prokaryotic or eukaryotic cells, culturing of complex tissues and organs, production of biological materials such as proteins, metabolites, therapeutics and the like, and similar applications, as it is well known in the art.

Any of a wide variety of cells, tissues, and the like can be grown, including but not limited to primary cell cultures, immortalized cell cultures, cultured cells, organs, tissues, etc. In some embodiments, the cell culture medium can be added to the interior compartment of the container and then inoculated with a cell culture. Alternatively, in some embodiments, the cell culture medium can be pre-inoculated with cells and then housed into the interior compartment of the bioprocessing container. Once the culture medium and cells have been deposited into the interior compartment, the cells can be incubated within the interior of the container under conditions suitable for cell growth (i.e., temperature, agitation, pH, and the like). Suitable conditions for each particular cell type are well known to those skilled in the art or can be ascertained using routine experimentation.

The disclosed bioprocessing container can be used also for processes other than cell growth, for example media preparation, buffer preparation, storage or processing of foods, chemicals, biopharmaceuticals, biologicals, and the like, storage of biological fluids such as serum, buffers, ultrapure water, blood, physiological solutions, etc).

### V. Advantages of the Disclosed Film

The disclosed film can be used to provide an improved bioprocessing container for e.g. growing cells in vitro. The disclosed film is biologically inert, as the surface of its sealant layer (which finally forms the internal surface of the bioprocessing container, which contacts the hosted material) has low interaction and does not interfere with the material inside the bioreactor (medium, cultured cells, products from the bioprocessing, housed biological fluids, etc.). Inertness favors the yield of cell growth, the harvesting of non-adherent cultured cells and of products from the bioprocessing and avoids contamination of the material inside the bioreactor. To this aim, the disclosed film substantially lacks migratory surface additives and metal containing particulate additives, that can interfere with cell growth or products collection. In addition, the sealant layer of the film provides a high seal strength even if the film is exposed to high temperatures. This allows the use of the disclosed film to manufacture bioreactors with a high load-carrying capacity and/or capable of facing internal pressure when in use, without the risk of leaking or seal rupture. Finally, the composition of the sealant layer avoids adhesion between the plies when the film is flattened after being manufactured as a tube. This permits the manufacture of the film in tubular form, which is advantageous because limits the risk of contamination of such layer.

The disposable flexible bioprocessing container manufactured with the disclosed film allows a user to operate the cell growth or processing processes with relative ease and little training.

Such disposable flexible bioprocessing container can be single-use, thus does not require cleaning or sterilizing after use, thereby preserving user time and resources.

Although several advantages of the disclosed film and bioprocessing container are set forth herein, the list is by no means limiting. Anyone skilled in the art can recognize that there can be several advantages to the disclosed film, bioprocessing container and bioprocessing method that are not mentioned herein.

### EXAMPLES

The following examples are presented for the purpose of further illustrating and explaining the presently disclosed subject matter and are not to be taken as limiting in any regard. In the light of the present disclosure and the general level of skill in the art, those skilled in the art will appreciate that the following Examples are intended to be exemplary only and that numerous changes, modifications, and alterations can be employed without departing from the scope of the presently disclosed subject matter. Unless otherwise indicated, all parts and percentages are by weight.

Table 1 reports the polymers used for manufacturing the films of the examples (according to the disclosed subject matter) and of the comparative examples.

TABLE 1
Polymers

| Tradename | Supplier | Acronym |
|---|---|---|
| EXACT 3024 | ExxonMobil | VLDPE1 |
| CV77539 | Westlake Chemical | LLDPE1 |
| SCLAIR 2908 | Nova Chemicals | HDPE1 |
| MB50-802 | DuPont | Silox MB1 |
| MB25-235 | DuPont | Silox MB2 |
| DOWLEX 2045.03 | Dow | LLDPE2 |
| XUS 61520.15L | Dow | VLDPE2 |
| EMAC SP2202 | Westlake Chemical | EBA1 |
| BYNEL 4164 | Dow | LLDPE-md1 |
| BYNEL 21E810 | Dow | EMA-md1 |
| EVASIN EV3251F | Chang Chun Petrochemicals Ltd. | EVOH1 |
| BYNEL 21E787 | Dow | EMA-md2 |
| Ecdel 9965 | Eastman Chemical | COPE1 |
| Ecdel 9965 ED005 | Eastman Chemical | Antiblock MB1 |
| AntiBlock | | |
| Topas E-140 | Topas Advanced Polymers, Inc. | COC1 |
| EMAC + SP1330 | Westlake Chemical | EMA1 |
| Bynel 46E1060 | Dow | VLDPE-md1 |
| ARNITEL VT3104 | DSM | PBTG1 |
| ARNITEL EM400 | DSM | PBTG2 |
| Siloxane MB50-010 | Dow Corning | Silox MB2 |
| 1000563-NP | Ampacet | LLDPE3 |
| CV77525 | Westlake Chemical | VLDPE3 |
| SP2260 | Westlake Chemical | EMA2 |
| TYMAX GT4409 | Westlake Chemical | LLDPE-md2 |
| BYNEL 3861 | Dow | EVA-md1 |
| SOARNOL ET3803 | Mitsubishi Chemical Corporation | EVOH2 |
| Eval L171B | EVALCA/Kuraray | EVOH3 |
| GRILON CF6S | EMS-Grivory | PA1 |
| Topas 8007F-600 | Topas Advanced Polymers, Inc. | COC2 |
| EVASIN EV3851F | Chang Chun Petrochemicals Ltd. | EVOH4 |
| Topas 7010F-600 | Topas Advanced Polymers, Inc. | COC3 |
| EV2951F | Chang Chun Petrochemicals Ltd. | EVOH5 |

VLDPE1: Very Low Density Polyethylene; Density 0.905 g/cc; Melt flow rate (190° C./2.16 kg) 4.5 g/10 min; Melting point: 97° C.; Vicat Softening Point 87° C.
LLDPE1: Linear Low Density Polyethylene (LLDPE); Density 0.910 g/cc; Melt flow rate (190° C./2.16 kg) 1.95 g/10 min; Melting point: 124° C.
HDPE1: Polyethylene High Density Homopolymer; Density 0.961 g/cc; Melt flow rate (190° C./2.16 kg) 7.0 g/10 min; Vicat Softening Point 129° C.
Silox MB1: Polydimethylsiloxane (slip) in LDPE; additive (High Molecular Weight Siloxane) content 50%; Density 1.03 g/cc; Melt flow rate (190° C./2.16 kg) 8 g/10 min.
Silox MB2: Polydimethylsiloxane (slip) in LDPE; additive (High Molecular Weight Siloxane) content 25%; Density 0.929 g/cc; Melt flow rate (190° C./2.16 kg) 5.2 g/10 min.
LLDPE2: Linear Low Density Polyethylene; Density 0.920 g/cc; Melt flow rate (190° C./2.16 kg) 1.10 g/10 min; Melting point: 124.5° C.
VLDPE2: Very Low Density Polyethylene; Density 0.903 g/cc; Melt flow rate (190° C./2.16 kg) 0.5 g/10 min; Melting point: 122° C.; Vicat Softening Point 100° C.
EBA1: Ethylene/Butyl Acrylate Copolymer; Density 0.943 g/cc; Melt flow rate (190° C./2.16 kg) 0.45 g/10 min; Melting point: 82° C.; Vicat Softening Point 53° C.
LLDPE-md1: Linear Low Density Maleic Anhydride-Modified Polyethylene; Density 0.930 g/cc; Melt flow rate (190° C./2.16 kg) 1.20 g/10 min; Melting point: 127° C.; Vicat Softening Point 109° C.
EMA-md1: Maleic Anhydride-Modified Ethylene/Methyl Acrylate Copolymer; Density 0.931 g/cc; Melt flow rate (190° C./2.16 kg) 2.20 g/10 min; Melting point: 94° C.
EVOH1: Hydrolyzed Ethylene/Vinyl Acetate Copolymer, Comonomer content (Ethylene) 32%; Density 1.19 g/cc; Melt flow rate (190° C./2.16 kg) 1.60 g/10 min; Melting point: 183° C.
EMA-md2: Maleic Anhydride-Modified Ethylene/Methyl Acrylate Copolymer; Density 0.930 g/cc; Melt flow rate (190° C./2.16 kg) 1.60 g/10 min; Melting point: 92° C.; Vicat Softening Point 52° C.
COPE1: Copolyester Ether Elastomer; Density 1.13 g/cc; Melt flow rate 19 g/10 min; Melting point 205° C.
Antiblock MB1: Antiblock and Antioxidant in Copolyester; Additives (Irganox1010) content 91000 ppm: Melting point 207° C.
COC1: Cyclic Olefin Copolymer (Ethylene/Norbomene Copolymer); Density 0.94 g/cc; Melt flow rate (190° C./2.16 kg) 3.00 g/10 min.
EMA1: Ethylene/Methyl Acrylate Copolymer; Comonomer (Acrylic Acid) content 22%; Density 0.944 g/cc; Melt flow rate (190° C./2.16 kg) 2.00 g/10 min; Melting point: 93° C.
VLDPE-md1: Maleic Anhydride-Modified Very Low Density Polyethylene; Density 0.87 g/cc; Melt flow rate (190° C./2.16 kg) 3 g/10 min; Melting point: 62.8° C.; Vicat Softening Point 40° C.
PBTG1: Polybutylene Terephthalate/Glycol Block Copolymer; Density 1.16 g/cc; Melt flow rate (230° C./2.16 kg) 13.5 g/10 min; Melting point: 212° C.
PBTG2: Polybutylene Terephthalate/Glycol Block Copolymer; Density 1.11 g/cc; Melt flow rate (230° C./2.16 kg) 34 g/10 min; Melting point: 195° C.
LLDPE3: Linear Low Density Polyethylene and Cyclic Olefin Copolymer with Tg≥25° C. (compounded polymer blend); Additives (Irganox1010) 380 ppm; Density 0.929 g/cc; Melt flow rate (190° C./2.16 kg) 4.5 g/10 min.
VLDPE3: Very Low Density Polyethylene; Additives (Irganox1076) 200 ppm; Density 0.906 g/cc; Melt flow rate (190° C./2.16 kg) 0.45 g/10 min; Melting point: 122° C.
EMA2: Ethylene/Methyl Acrylate Copolymer; Comonomer (MethylAcrylate) content 24%; Additives (Irganox1076) 275 ppm; Additives (BHT) 550 ppm; Density 0.944 g/cc; Melt flow rate (190° C./2.16 kg) 2.1 g/10 min; Melting point: 76° C.
LLDPE-md2: Linear Low Density Maleic Anhydride-Modified Polyethylene; Additives (Irgafos168) 145 ppm; Additives (Irganox1076) 750 ppm; Density 0.919 g/cc; Melt flow rate (190° C./2.16 kg) 2 g/10 min; Melting point: 124° C.
EVA-md1: Maleic Anhydride-Modified Ethylene/Vinyl Acetate Copolymer; Comonomer (VinylAcetate) content 25%; Density 0.950 g/cc; Melt flow rate (190° C./2.16 kg) 2 g/10 min.
EVOH2: Hydrolyzed Ethylene/Vinyl Acetate Copolymer, Comonomer content (Ethylene) 38%; Density 1.17 g/cc; Melt flow rate (210° C./2.16 kg) 3.3 g/10 min; Melting point: 179° C.; Glass Transition Temperature: 58° C.
EVOH3: Hydrolyzed Ethylene/Vinyl Acetate Copolymer, Comonomer content (Ethylene) 27%; Density 1.21 g/cc; Melt flow rate (210° C./2.16 kg) 4 g/10 min; Melting point: 190° C.; Glass Transition Temperature: 60° C.
PA1: Polyamide 6/12; Density 1.05 g/cc; Melt flow rate (190° C./5.00 kg) 5.75 g/10 min; Melting point: 130° C.
COC2: Cyclic Olefin Copolymer (Ethylene/Norbornene Copolymer); Density 1.02 g/cc; Melt flow rate (190° C./2.16 kg) 2.04 g/10 min.
EVOH4: Hydrolyzed Ethylene/Vinyl Acetate Copolymer, Comonomer content (Ethylene) 38%; Density 1.17 g/cc; Melt flow rate (190° C./2.16 kg) 1.80 g/10 min; Melting point: 171° C.; Glass Transition Temperature: 54° C.
COC3: Cyclic olefinic copolymer; Density 1.02 g/cc; Melt flow rate (190° C./2.16 kg) 0.816 g/10 min; Glass Transition Temperature: 110° C.
EVOH5: Hydrolyzed Ethylene/Vinyl Acetate Copolymer, Comonomer content (Ethylene) 29%; Density 1.21 g/cc; Melt flow rate (210° C./2.16 kg) 3.00 g/10 min; Melting point: 187° C.

TABLE 2
Films and Comparative Films

| | | | Layer | Film |
|---|---|---|---|---|
| | | | thickness | thickness |
| Film | Layer | Composition | (microns) | (microns) |
| Film 1 | 1 | 68% VLDPE1 | 20.3 | 314.8 |
| | | 24% LLDPE1 | | |
| | | 5.6% HDPE1 | | |
| | | 2.4% Silox MB1 | | |
| | 2 | 70% VLDPE1 | 45.7 | |
| | | 30% LLDPE1 | | |
| | 3 | 30% LLDPE2 | 55.9 | |
| | | 55% VLDPE2 | | |
| | | 15% EBA1 | | |
| | 4 | 70% LLDPE-md1 | 20.3 | |
| | | 30% EMA-md1 | | |
| | 5 | 100% EVOH1 | 15.2 | |
| | 6 | 70% LLDPE-md1 | 20.3 | |
| | | 30% EMA-md1 | | |
| | 7 | 100% EVOH1 | 15.2 | |
| | 8 | 70% LLDPE-md1 | 20.3 | |
| | | 30% EMA-md1 | | |
| | 9 | 30% LLDPE2 | 66 | |
| | | 55% VLDPE2 | | |
| | | 15% EBA1 | | |
| | 10 | 100% EMA-md2 | 12.7 | |
| | 11 | 95% COPE1 | 22.9 | |
| | | 5% Antiblock MB1 | | |
| Film 2 | 1 | 56% VLDPE1 | 20.3 | 315 |
| | | 24% LLDPE1 | | |
| | | 12% COC1 | | |
| | | 5.6% HDPE1 | | |
| | | 2.4% Silox MB1 | | |
| | 2 | 70% VLDPE1 | 45.7 | |
| | | 30% LLDPE1 | | |
| | 3 | 40% LLDPE2 | 55.9 | |
| | | 50% VLDPE2 | | |
| | | 10% EMA1 | | |
| | 4 | 55% LLDPE2 | 20.3 | |
| | | 45% VLDPE-md1 | | |
| | 5 | 100% EVOH1 | 15.2 | |
| | 6 | 55% LLDPE2 | 20.3 | |
| | | 45% VLDPE-md1 | | |
| | 7 | 100% EVOH1 | 15.2 | |
| | 8 | 55% LLDPE2 | 20.3 | |
| | | 45% VLDPE-md1 | | |
| | 9 | 40% LLDPE2 | 66 | |
| | | 50% VLDPE2 | | |
| | | 10% EMA1 | | |
| | 10 | 100% EMA-md2 | 15.2 | |
| | 11 | 95% PBTG1 | 20.3 | |
| | | 2% Silox MB2 | | |
| | | 3% Antiblock MB1 | | |
| Film 3 | 1 | 69% VLDPE1 | 43.2 | 304.8 |
| | | 25% LLDPE1 | | |
| | | 4.2% HDPE1 | | |
| | | 1.8% Silox MB1 | | |
| | 2 | 70% VLDPE2 | 109.2 | |
| | | 30% VLDPE1 | | |
| | 3 | 75% VLDPE2 | 88.9 | |
| | | 25% VLDPE-md1 | | |
| | 4 | 100% EVOH1 | 31.8 | |
| | 5 | 100% EMA-md1 | 12.7 | |
| | 6 | 97% PBTG1 | 19 | |
| | | 3% Silox MB2 | | |
| Film 4 | 1 | 90% VLDPE1 | 25.4 | |
| | | 6% HDPE1 | | |
| | | 4% Silox MB2 | | |
| | 2 | 70% VLDPE2 | 107.9 | |
| | | 30% VLDPE1 | | |
| | 3 | 65% LLDPE-md1 | 19.0 | |
| | | 35% EMA-md1 | | |
| | 4 | 100% EVOH1 | 15.2 | |
| | 5 | 65% LLDPE-md1 | 19.0 | |
| | | 35% EMA-md1 | | |
| | 6 | 100% EVOH1 | 15.2 | |
| | 7 | 65% LLDPE-md1 | 19.0 | |
| | | 35% EMA-md1 | | |
| | 8 | 70% VLDPE2 | 61.0 | |
| | | 30% VLDPE1 | | |
| | 9 | 100% EMA-md1 | 15.2 | |
| | 10 | 95% PBTG2 | 20.3 | |
| | | 3% Antiblock MB1 | | |
| | | 2% Silox MB2 | | |
| Comparative | 1 | 100% LLDPE3 | 88.9 | 330.2 |
| Film 1 | 2 | 50% VLDPE3 | 63.5 | |
| | | 50% EMA2 | | |
| | 3 | 50% LLDPE-md2 | 12.7 | |
| | | 50% EVA-md1 | | |
| | 4 | 100% EVOH2 | 12.7 | |
| | 5 | 50% LLDPE-md2 | 12.7 | |
| | | 50% EVA-md1 | | |
| | 6 | 100% EVOH3 | 12.7 | |
| | 7 | 50% LLDPE-md2 | 12.7 | |
| | | 50% EVA-md1 | | |
| | 8 | 50% VLDPE3 | 88.9 | |
| | | 50% EMA2 | | |
| | 9 | 50% LLDPE-md2 | 12.7 | |
| | | 50% EVA-md1 | | |
| | 10 | 96% PA1 | 12.7 | |
| | | 4% Antiblock MB1 | | |
| Comparative | 1 | 85% LLDPE1 | 88.9 | 330.2 |
| Film 2 | | 15% COC2 | | |
| | 2 | 50% VLDPE3 | 63.5 | |
| | | 50% EMA2 | | |
| | 3 | 50% LLDPE-md2 | 12.7 | |
| | | 50% EVA-md1 | | |
| | 4 | 100% EVOH4 | 12.7 | |
| | 5 | 50% LLDPE-md2 | 12.7 | |
| | | 50% EVA-md1 | | |
| | 6 | 100% EVOH1 | 12.7 | |
| | 7 | 50% LLDPE-md2 | 12.7 | |
| | | 50% EVA-md1 | | |
| | 8 | 50% VLDPE3 | 88.9 | |
| | | 50% EMA2 | | |
| | 9 | 50% LLDPE-md2 | 12.7 | |
| | | 50% EVA-md1 | | |
| | 10 | 96% PA1 | 12.7 | |
| | | 4% Antiblock MB1 | | |
| Comparative | 1 | 80% LLDPE1 | 88.9 | 330.2 |
| Film 3 | | 10% COC2 | | |
| | | 10% COC3 | | |
| | 2 | 50% VLDPE3 | 63.5 | |
| | | 50% EMA2 | | |
| | 3 | 50% LLDPE-md2 | 12.7 | |
| | | 50% EVA-md1 | | |
| | 4 | 100% EVOH4 | 12.7 | |
| | 5 | 50% LLDPE-md2 | 12.7 | |
| | | 50% EVA-md1 | | |
| | 6 | 100% EVOH5 | 12.7 | |
| | 7 | 50% LLDPE-md2 | 12.7 | |
| | | 50% EVA-md1 | | |
| | 8 | 50% VLDPE3 | 88.9 | |
| | | 50% EMA2 | | |
| | 9 | 50% LLDPE-md2 | 12.7 | |
| | | 50% EVA-md1 | | |
| | 10 | 96% PA1 | 12.7 | |
| | | 4% Antiblock MB1 | | |
| Comparative | 1 | 87% LLDPE1 | 88.9 | 330.2 |
| Film 4 | | 13% COC2 | | |
| | 2 | 50% VLDPE3 | 63.5 | |
| | | 50% EMA2 | | |
| | 3 | 50% LLDPE-md2 | 12.7 | |
| | | 50% EVA-md1 | | |
| | 4 | 100% EVOH4 | 12.7 | |
| | 5 | 50% LLDPE-md2 | 12.7 | |
| | | 50% EVA-md1 | | |
| | 6 | 100% EVOH5 | 12.7 | |
| | 7 | 50% LLDPE-md2 | 12.7 | |
| | | 50% EVA-md1 | | |
| | 8 | 50% VLDPE3 | 88.9 | |
| | | 50% EMA2 | | |
| | 9 | 50% LLDPE-md2 | 12.7 | |
| | | 50% EVA-md1 | | |
| | 10 | 96% PA1 | 12.7 | |
| | | 4% Antiblock MB1 | | |

### Film Manufacture

Films 1-4 and Comparative Films 1-4 were all manufactured by round cast coextrusion. This extrusion method is well known to those of ordinary skill in the art.

### Maximum Seal Force Measurement

The maximum seal force (force required to open the seals formed between two specimens of a film) was measured for Film 1 and for Comparative Films 1, 2, 3, and 4, according to ASTM-F88, at a slow speed of 0.254 cm/min (0.10 inch/min). The maximum seal force was measured on non-aged film samples and on film samples that underwent accelerated aging, i.e., that were conditioned at 60° C. for 24 hours before testing

For each of Film 1 and Comparative Films 1, 2, 3 and 4, three (3) specimens having dimensions of 2.54 cm (1.00 in) in width and about 7.62 cm (3.00 in) in length were cut in the machine direction, and three specimens having the same dimensions were cut in the transverse direction. The specimens were sealed on themselves with the sealant layers facing each other. The heat-sealed films were manually produced using a Packworld PW3124 two-sided impulse sealer.

Sealing occurred in the machine direction (three specimens) or in the transverse direction (three specimens) with a sealing dwell time of 12 seconds and a sealing temperature of 160° C.

The specimens of the sealed films were manually separated in order to provide detached film portions sufficient to be fixed into the lower jaw and into the upper jaw of a dynamometer. The area to be tested was positioned in the middle of the two jaws of the dynamometer, and adequate pre-tensioning between the two extremities of the fixed sample was applied.

The instrument measured the maximum force needed to separate the two sealed films. In particular, it measured the force (N/25 mm) applied for the opening of 2.54 cm (width of seal) of the seal for each specimen.

The maximum seal force was measured on the 3 specimens cut and sealed in the machine direction and on the 3 specimens cut and sealed in the transverse direction. The combined average seal force was calculated on the 6 specimens (3 cut and sealed in the machine direction and 3 cut and sealed in the transverse direction). The combined average seal force and the standard deviations are reported in Table 3.

The dynamometer conditions were:
Equipment: Instron 5564
Starting jaw distance: 2.54 cm (1.00 in)
Crosshead speed: 0.254 mm/min (0.10 in)
Seal length: 2.54 cm (1.00 in)
Seal width: 1 cm (0.39 in)

TABLE 3
Maximum seal force

| | | | Accelerated aged | |
|---|---|---|---|---|
| | | | samples | |
| | Non-aged samples | | (60° C., 24 hrs.) | |
| | Average | Standard | Average | Standard |
| | (N/25 mm) | Deviation | (N/25 mm) | Deviation |
| Comparative | 52.76 | 1.06 | 40.01 | 3.95 |
| Film 1 | | | | |
| Comparative | 56.42 | 1.13 | 44.90 | 2.29 |
| Film 2 | | | | |
| Comparative | 39.72 | 3.3 | 19.18 | 2.06 |
| Film 3 (*) | | | | |
| Comparative | 60.46 | 4.5 | 43.7 | 5.5 |
| Film 4 | | | | |
| Film 1 | 94.00 | 0.67 | 94.53 | 1.36 |

(*) sealing temperature was 200° C.

Based on the data, Film 1 shows a remarkably higher maximum seal strength than the comparative films. The higher maximum seal strength of Film 1 with respect to the comparative films is even more evident after accelerated aging (i.e., exposure to 60° C. for 24 hours). Notably, Film 1 shows perfectly comparable maximum seal strength values when tested non-aged and after accelerated aging.

### Blocking Effect

After manufacture in tubular form, Films 1-4 were flattened to form a two-ply flattened tube with the sealant layers of the two plies contacting each other towards the inside of the tube. Upon plies separation, very low to no blocking effect was noted, and plies separated in a smooth and even way.

All references to (and incorporations by reference of) ASTM protocols are to the most-recently published ASTM procedure as of the priority (i.e., original) filing date of this patent application unless stated otherwise.

1. A polymeric, multilayer film comprising:
a sealant layer comprising:
(i) 58-95.5 wt % in respect of the total weight of the sealant layer of one or more ethylene/alpha olefin copolymers having an average density lower than 0.922 g/cc;
(ii) 4-10 wt % in respect of the total weight of the sealant layer of one or more polymers having an average density higher than or equal to 0.950 g/cc and selected among one or more ethylene/alpha olefin copolymers and/or one or more ethylene homopolymers;
(iii) 0.5-2.0 wt % in respect of the total weight of the sealant layer of high molecular weight siloxane polymers;
an outer skin layer comprising one or more polymers selected from the group consisting of: (co)polyesters, polyamides; high-density polyethylene (HDPE), polypropylene (PP) and their admixtures.

2. The polymeric, multilayer film of claim 1 wherein the one or more ethylene/alpha olefin copolymers having an average density lower than 0.922 g/cc are selected from the group consisting of linear low density polyethylene (LLDPE), very low density polyethylene (VLDPE), ultra low density polyethylene (ULDPE) and mixtures thereof.

3. The polymeric, multilayer film of claim 1 wherein the one or more polymers having an average density higher than or equal to 0.950 g/cc is high density polyethylene (HDPE) or mixtures thereof.

4. The polymeric, multilayer film of claim 1 comprising 75-95.5 wt % in respect of the total weight of the sealant layer of one or more ethylene/alpha olefin copolymers having an average density lower than 0.922 g/cc.

5. The polymeric, multilayer film of claim 1 comprising:
(i) 75-93.5 wt % in respect of the total weight of the sealant layer of one or more ethylene/alpha olefin copolymers having an average density lower than 0.922 g/cc;
(ii) 6-10 wt % in respect of the total weight of the sealant layer of one or more polymers having an average density higher than or equal to 0.950 g/cc and selected among one or more ethylene/alpha olefin copolymers and/or one or more ethylene homopolymers;
(iii) 0.5 to 1.2 wt % in respect of the total weight of the sealant layer of high molecular weight siloxane polymers.

6. The polymeric, multilayer film of claim 1 comprising:
(i) 75-95.2 wt % in respect of the total weight of the sealant layer of one or more ethylene/alpha olefin copolymers having an average density lower than 0.922 g/cc;
(ii) 4-6 wt % in respect of the total weight of the sealant layer of one or more polymers having an average density higher than or equal to 0.950 g/cc and selected among one or more ethylene/alpha olefin copolymers and/or one or more ethylene homopolymers; and
(iii) 0.8 to 2.0 wt % in respect of the total weight of the sealant layer of high molecular weight siloxane polymers.

7. The polymeric, multilayer film of claim wherein each one of the at least one gas barrier layer(s), if present, independently comprises a major proportion of one or more polymers selected from the group consisting of poly(glycolic acid) (PGA), polyamides, ethylene vinyl alcohol (EVOH) and mixtures thereof.

8. The polymeric, multilayer film of claim 1 wherein the outer skin layer comprises a major proportion of one or more polymers selected from the group consisting of (co)polyesters, polyamides, high-density polyethylene (HDPE), polypropylene (PP) and their admixtures.

9. The polymeric, multilayer film of claim 8 wherein the outer skin layer comprises a major proportion of one or more polymers selected from the group consisting of: (co)polyesters, polyamides, and their admixtures.

10. The polymeric, multilayer film of claim 1 further comprising:
one or more bulk layer independently comprising one or more polymers selected from the group consisting of low density polyethylene (LDPE), ethylene-vinyl acetate copolymers (EVA), linear low density polyethylenes (LLDPE), linear very low density polyethylenes (VLDPE), ethylene/acrylic acid copolymers (EAA), ethylene/methacrylic acid copolymers (EMAA), ethylene/methyl acrylate copolymers (EMA), ethylene/butyl acrylate copolymers (EBA), ionomers, and mixtures thereof;
and/or
one or more tie layer independently comprising one or more polymers selected from the group consisting of ethylene-unsaturated acid copolymers, ethylene-unsaturated ester copolymers, polyurethane, ethylene-vinyl acetate copolymers, ethylene-(meth)acrylic acid copolymers, ethylene/methyl acrylate copolymers, ethylene homo-polymers or co-polymers modified with anhydride or carboxylic acid functionalities, mixtures thereof and mixtures of any of these resins with an ethylene homo- or co-polymer, anhydride modified grafted linear low density polyethylene, anhydride grafted low density polyethylene, homogeneous ethylene/alpha-olefin copolymer, anhydride grafted ethylene/vinyl acetate copolymer, anhydride grafted ethylene/methyl acrylate copolymers, anhydride grafted styrene/ethylene/butadiene/styrene copolymers.

11. The polymeric, multilayer film of claim 1 having a thickness comprised between 30 and 600 microns.

12. The polymeric, multilayer film of claim 1 wherein the film comprises migratory surface additives and from metal containing particulate additives in a total amount of 0.05 wt % or less, based on the total weight of the film.

13. A method for manufacturing the film of claim 1 comprising a step of extruding all the layers of the film, or at least some layers thereof, comprising the sealant layer, through a round die.

14. A flexible bioprocessing container comprising at least a polymeric, multilayer film comprising:
a sealant layer comprising:
(i) 58-95.5 wt % in respect of the total weight of the sealant layer of one or more ethylene/alpha olefin copolymers having an average density lower than 0.922 g/cc;
(ii) 4-10 wt % in respect of the total weight of the sealant layer of one or more polymers having an average density higher than or equal to 0.950 g/cc and selected among one or more ethylene/alpha olefin copolymers and/or one or more ethylene homopolymers;
(iii) 0.5-2.0 wt % in respect of the total weight of the sealant layer of high molecular weight siloxane polymers;
an outer skin layer comprising one or more polymers selected from the group consisting of: (co)polyesters, polyamides; high-density polyethylene (HDPE), polypropylene (PP) and their admixtures,
wherein said film is sealed to define an interior compartment of the container and wherein the sealant layer of said film forms an inner surface of the container.

15. The flexible bioprocessing container of claim 14, wherein the container is pre-sterilized.

16. A method for culturing cells comprising either the steps of:
(i) providing a flexible bioprocessing container;
(ii) introducing a liquid medium into the interior compartment of the container;
(iii) inoculating the liquid medium with cells, and
(iv) incubating the cells within the interior compartment of the container under suitable conditions for cell growth;
or the steps of:
(i) providing a flexible bioprocessing container;
(ii) pre-inoculating a liquid medium with cells;
(iii) introducing the pre-inoculated liquid medium into the interior compartment of the container, and
(iv) incubating the cells within the interior compartment of the container under suitable conditions for cell growth;
in either instance, the flexible bioprocessing container comprising at least a polymeric, multilayer film comprising:
a sealant layer comprising:
(i) 58-95.5 wt % in respect of the total weight of the sealant layer of one or more ethylene/alpha olefin copolymers having an average density lower than 0.922 g/cc;
(ii) 4-10 wt % in respect of the total weight of the sealant layer of one or more polymers having an average density higher than or equal to 0.950 g/cc and selected among one or more ethylene/alpha olefin copolymers and/or one or more ethylene homopolymers;
(iii) 0.5-2.0 wt % in respect of the total weight of the sealant layer of high molecular weight siloxane polymers;
an outer skin layer comprising one or more polymers selected from the group consisting of: (co)polyesters, polyamides; high-density polyethylene (HDPE), polypropylene (PP) and their admixtures.

17. The polymeric, multilayer film of claim 1 wherein the sealant layer further comprises (iv) 5-30 wt % in respect of the total weight of the sealant layer of elastomeric cyclic olefin copolymers (COC) with glass transition temperature (Tg) lower than 20° C., wherein the glass transition temperature is measured by differential scanning calorimetry (DSC) according to ASTM D-3418.

18. The polymeric, multilayer film of claim 1 wherein the at least one gas barrier layer comprises one or more polymers selected from the group consisting of poly(glycolic acid) (PGA), polyamides, ethylene vinyl alcohol (EVOH) and mixtures thereof.

19. The polymeric, multilayer film of claim 5 wherein the ethylene/alpha olefin copolymers having an average density lower than 0.922 g/cc are selected among LLDPE, VLDPE, ULDPE and mixtures thereof, and/or the one or more polymers having an average density higher than or equal to 0.950 g/cc is HDPE or mixtures thereof.

20. The polymeric, multilayer film of claim 9 wherein the one or more polymers selected from the group consisting of: (co)polyesters, polyamides, and their admixtures of the outer skin layer is polybutylene terephthalate (PBT) and copolymers thereof, and wherein such PBT copolymer is a copolymer of terephthalic acid, butylene glycol and a further glycol (PBTG).
